(19) United States
(12) Patent Application Publication
(10) Pub. No.: US 2021/0298860 A1
(43) Pub. Date: Sep. 30, 2021

(54) MEDICAL SUPPORT SLEEVE DEVICES, SYSTEMS AND METHODS

(71) Applicant: Radux Devices, LLC, Omaha, NE (US)

(72) Inventor: Gregory Gordon, Omaha, NE (US)

(21) Appl. No.: 17/264,712

(22) PCT Filed: Jul. 24, 2019
(86) PCT No.: PCT/US2019/043200

§ 371 Date: Jan. 29, 2021

Related U.S. Application Data
(63) Continuation of application No. 16/049,471, filed on Jul. 30, 2018, now abandoned.
(60) Provisional application No. 62/748,858, filed on Oct. 22, 2018.

(51) Int. Cl. A61B 46/27; A61M 5/52

(52) U.S. Cl. CPC A61B 46/27; A61B 2046/201; A61M 5/52

(57) ABSTRACT

In general, medical sleeve systems that at least partially support a patient's arm in a selected position are described. In one embodiment, an example support sleeve includes a proximal end that defines an arm opening, a distal end, a wall that extends between the proximal end and the distal end, the wall including an anterior side portion and a posterior side portion, and an access port located on the anterior side portion proximate the distal end. The support sleeve may be manipulated between first and second positions, and at least partially maintained in the second position during a medical procedure.

### CROSS-REFERENCE TO RELATED APPLICATIONS

This application claims priority to U.S. Application Ser. No. 62/748,858, filed on Oct. 22, 2018, and U.S. application Ser. No. 16/049,471, filed on Jul. 30, 2018. The disclosure of the prior application is considered part of the disclosure of this application, and is incorporated in its entirety into this application.

### TECHNICAL FIELD

This document describes devices, systems and methods for supporting an arm during a medical procedure, and in some embodiments, supporting an arm during a medical procedure using a sterile, disposable support sleeve.

### BACKGROUND

Many interventional access procedures of a patient's arm involve stabilization of the arm. Various techniques have been developed that attempt to immobilize the arm during the procedure. In some procedures, the arm may be moved between multiple positions throughout the course of the procedure. For example, the arm may be in a first position during introduction of an interventional tool, and a second position while the interventional tool is manipulated through the patient's vasculature. Additionally, a radiation field is often employed to facilitate navigation of an interventional tool, monitor the patient's vasculature, and/or otherwise visualize one or more portions of the patient's anatomy. Radiation protection in the medical field is important, particularly for healthcare practitioners. Procedures and therapies are often designed to minimize patient exposure while allowing healthcare practitioners to effectively treat the patient. However, cumulative exposure of healthcare practitioners may be significant as they often perform multiple treatments in a typical day, and may be increased depending on where the healthcare practitioner may be required to stand relative to the radiation source in order to effectively conduct the procedure.

### SUMMARY

Some embodiments described herein include devices, systems and methods that facilitate vascular access of a patient and efficient operating room workflow during a medical procedure. Some example embodiments may include a support sleeve having a proximal end that defines an arm opening, a distal end, and a tubular wall of material that extends between the proximal and distal ends. The support sleeve may include an access opening proximate the distal end, and may be positioned over the patient's arm to provide a sterile operating field. The access opening defines a passage that an interventional tool may be passed through to access the patient's vasculature, such as a radial artery, during the medical procedure. The support sleeve may support the patient's arm in a selected position, while facilitating movement between multiple positions.

In various optional embodiments, the support sleeve includes one or more attachment locations configured to stabilize the support sleeve (e.g., and patient's arm within the support sleeve) relative to a substrate. For example, in some embodiments, the support sleeve includes an attachment device attachable to a substrate separate from the support sleeve, such as a strap, belt, drape, etc. associated with the patient, operating table, or other substrate in the operating room. In an example embodiment, the attachment device is configured to at least partially stabilize the patient's arm in a pronated or palm-down position on the patient's torso.

In some example embodiments, the support sleeve facilitates manipulation of the patient's arm between a first position, such as a supinated or palm-up position in which the vasculature can be accessed through the anterior side of the patient's arm, to a second position, such as a pronated or palm-down position in which an interventional procedure can be carried out. In some embodiments, the support sleeve may facilitate a radial access procedure in which the left arm of the patient is accessed, and at least a portion of the interventional procedure carried out by a healthcare practitioner operating from the right side of the patient, or vice versa (e.g., while the patient's arm rests on the patient's torso in a pronated position).

In various optional embodiments, an example medical sleeve system includes a substrate that the sleeve is attachable with. For example, the substrate may be a surgical drape, belt, band, strap, etc. In some optional embodiments, the substrate may include one or more complementary attachment devices, such as an adhesive, hook-and-loop fastener (e.g., the substrate may include the loop side and the support sleeve may include the hook side of hook-and-loop fastener, or vice versa), snaps, straps, ties, frictional materials, self-adherent non-woven materials, self-adherent polymer materials, coadhesive materials, highly frictional polymer, etc.

The support sleeve may be brought into engagement with the substrate in a selected orientation, and readily attached with the substrate to at least partially support the patient's arm in the selected orientation.

Particular embodiments described herein include a medical sleeve system. The medical sleeve system includes a support sleeve including a proximal end that defines an arm opening, a distal end, a wall that extends between the proximal end and the distal end, the wall including an anterior side portion and a posterior side portion, and an access port located on the anterior side portion proximate the distal end. The system further includes an attachment device configured to attach the wall of the support sleeve to a substrate.

In some implementations, the system may optionally include one or more of the following features. The attachment device may include an adhesive. The attachment device may be selected from the group consisting of hook-and-loop fastener, snaps, hooks, straps, ties, self-adherent non-woven material, self-adherent polymer material, highly frictional polymer material, coadhesive material, etc.

The attachment device may be located on the anterior side portion of the wall. The medical sleeve system may further include a stabilization substrate, wherein the attachment device is located on the stabilization substrate. The attachment substrate may include a surgical drape configured to extend at least partially around a patient's torso. The stabilization substrate may include a belt. The stabilization substrate may include a strap configured to extend around a patient's neck. A central longitudinal axis may extend centrally between proximal and distal ends of the access port, and the attachment device may be laterally offset from the central longitudinal axis of the access port. The attachment device may include two or more snaps that are laterally offset from the central longitudinal axis of the access port. The access port may be covered by a removable liner. An adhesive may at least partially surround the access port on an interior surface of the anterior side portion, the adhesive may be configured to adhere to a patient's skin when the tubular wall is positioned around the patient's arm. The wall may be made from a fluid impermeable material. The support sleeve may include a preformed articulated region that defines an angle between 10° and 90°. When the support sleeve is positioned on the patient's arm during use, the preformed articulated region may be configured to be located at an elbow location of the patient's arm while the access port is located at an inner wrist location of the patient's arm.

Particular embodiments described herein include a medical sleeve system. The medical sleeve system may include a support sleeve, including a proximal end that defines an arm opening, a distal end, a tubular wall that extends between the proximal end and the distal end, the tubular wall including an anterior side portion and a posterior side portion, an access port located on the anterior side portion proximate the distal end, and a first attachment device located on the anterior side portion of the tubular wall configured to attach the tubular wall of the support sleeve to a substrate. The medical sleeve system may further include a stabilization substrate separate from the support sleeve, the stabilization substrate including a second attachment device complementary to the first attachment device. The first attachment device is configured to be engaged with the second attachment device to at least partially support the support sleeve in a selected position.

In some implementations, the system may optionally include one or more of the following features. The first and second attachment devices comprise hook-and-loop fastener.

Particular embodiments described herein include a method of supporting an arm during medical procedure. The method includes positioning a support sleeve over a patient's arm, the support sleeve including a proximal end that defines an arm opening, a distal end, a wall that extends between the proximal end and the distal end, the wall including an anterior side portion and a posterior side portion, and an access port located on the anterior side portion proximate the distal end. The method further includes introducing a medical device through an access point in an anatomical vessel of a patient through the access port of the support sleeve while the patient's arm is in a first position, and supporting the patient's arm in a second position that is different from the first position, by engaging the support sleeve with a stabilization substrate.

In some implementations, the system may optionally include one or more of the following features. The support sleeve may include an attachment device located on the anterior side portion of the wall, and supporting the patient's arm in the second position may include engaging the attachment device with the stabilization substrate. The stabilization substrate may include an attachment device, and supporting the patient's arm in the second position may include engaging the attachment device with the support sleeve.

Some embodiments of the devices, systems and techniques described herein may provide one or more of the following advantages. First, some embodiments described herein may improve operating room workflow by reducing patient preparation time. The support sleeve may facilitate preparation of a sterile operating field. The access location may be prepped and the support sleeve readily positioned over the patient's arm, while the access port of the support sleeve facilitates vascular access (e.g., of the radial artery).

Second, some embodiments herein may improve operating room workflow by facilitating movement of the patient's arm between a first position and a second position. The first position may be a position beneficial for introduction of a medical device, such as a supinated or palm-up position in which the anterior side of the wrist is exposed and accessible. The second position may be a position beneficial for conducting at least a portion of the medical procedure, such as a pronated or palm-down position in which the wrist is resting on the patient's torso (e.g., near the inguinal region). A medical device can thus be readily introduced, while the arm can subsequently be manipulated (e.g., medially rotated) into a position that promotes patient comfort and facilitates ergonomic operation by the healthcare practitioner and/or operation by the healthcare practitioner from a location further from a radiation field.

Third, some embodiments herein facilitate efficient stabilization of a patient's arm in a selected orientation. For example, the support sleeve may optionally include one or more attachment devices that stabilize the patient's arm in a selected position, such as a pronated position in which the arm rests at least partially across the torso with the wrist near the inguinal region.

Fourth, some embodiments herein provide a sleeve system that is disposable after each use. A single-use system promotes sterility while reducing pre- and post-procedure sterilization techniques. The sleeve system can be cost-effectively manufactured for single-use, and/or safely disposed with surgical draping.

Fifth, some embodiments herein provide a sleeve system that is suitable for a variety of patient sizes and anatomy. A single sleeve configuration may be suitable for use over either the left or the right arm. Moreover, in some embodiments, the sleeve system may be adjustable or otherwise suitable for a range of arm sizes, such as by including stretchable material, adjustable straps, ties, snaps, etc., and/or other features.

Sixth, some embodiments herein facilitate particular medical procedures that involve radial artery access or vascular access via the wrist or arm, including coronary catheterization, percutaneous peripheral intervention, percutaneous coronary intervention, carotid artery and subclavian artery intervention for acute strokes, treatment of vascular malformations and aneurysms, central vein access for electrophysiology procedures, hemodialysis intervention, arteriovenous fistula (AVF) intervention, electrophysiological cardiology procedures, pacemaker insertions, mesenteric and renal arterial system, and/or other applications.

Seventh, some embodiments facilitate procedures involving left radial artery access with a healthcare practitioner operating from a right side of the patient, or vice versa. For example, some example sleeve systems facilitate stabilization of the patient's arm in a resting position across the patient's torso. The healthcare practitioner may manipulate an interventional tool through the access location of the patient's arm while standing in an upright position, which may reduce stress and fatigue on the practitioner, and/or at an extended distance from a radiation field. The operator's hands may be positioned outside of field of radiation during the operation, and the physician's head, neck and other body part's may be positioned a greater distance from a field of radiation. Alternatively, or additionally, the patient's arm may be stabilized in a selected position from which the healthcare practitioner may more comfortably manipulate an interventional tool in a natural, ergonomic position, and/or by using a dominant hand. Thus, the sleeve system may allow the healthcare practitioner to operate from a position selected primarily based on advantages in patient care, such as operating from the left radial artery in a percutaneous coronary intervention, while being less constrained by ergonomic requirements, for example.

The details of one or more implementations are set forth in the accompanying drawings and the description below. Other features and advantages will be apparent from the description and drawings, and from the claims.

### DESCRIPTION OF DRAWINGS

The present description is further provided with reference to the appended Figures, wherein like structure is referred to be like numerals throughout the several views, and wherein:

FIG. 1 is a perspective view of an example sleeve system in use in a medical environment.

FIG. 2 is a perspective view of the example sleeve system of FIG. 1.

FIG. 3 is a perspective view of the example sleeve system of FIG. 1 in which a patient's arm is in a first, supinated position.

FIG. 4 is a perspective view of the example sleeve system of FIG. 1 in which the patient's arm is in a second, pronated position.

FIG. 5A is a perspective view of another example sleeve system including multiple attachment devices in which a patient's arm is in a first, supinated position.

FIG. 5B is a perspective view of the example sleeve system of FIG. 5 in which the patient's arm is in a second, pronated position.

FIG. 6A is a perspective view of another example sleeve system in which the patient's arm is partially supinated.

FIG. 6B is a perspective view of the example sleeve system of FIG. 6 in which the patient's arm is in a pronated position.

FIG. 7 is perspective view of another example sleeve system having an expandable portion.

FIG. 8 is a flow diagram of an example process of using a sleeve system.

FIG. 9A is a perspective view of another example sleeve system.

FIG. 9Bb is a perspective view of the example sleeve system of FIG. 9A in which a patient's arm is in a first, supinated position.

FIG. 9C is a perspective view of the example sleeve system of FIG. 9A in which the patient's arm is in a second, pronated position.

FIG. 9D is a perspective view of another example sleeve system in which a patient's arm is in a first, supinated position.

FIG. 9E is a perspective view of the example sleeve system of FIG. 9D in which the patient's arm is in a second, pronated position.

### DETAILED DESCRIPTION OF ILLUSTRATIVE EMBODIMENTS

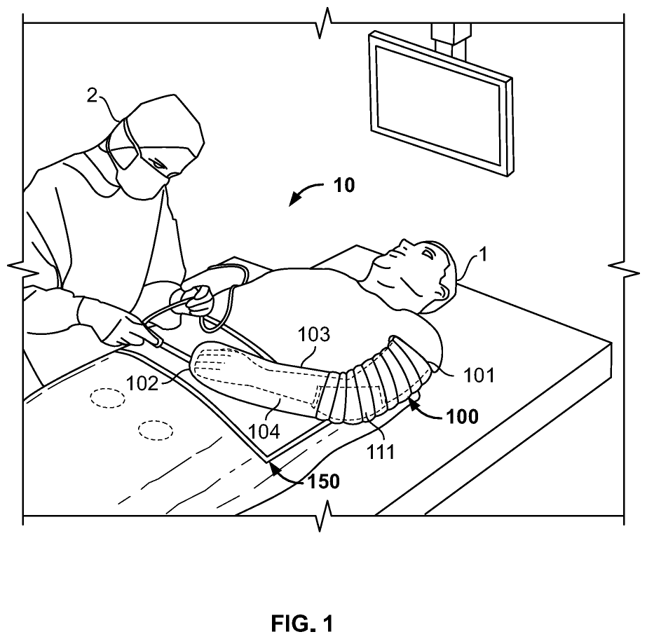

Referring to FIGS. 1-4, an example medical sleeve system 10 is shown, including a support sleeve 100, and optionally, a stabilization substrate 150 that the support sleeve 100 may be engaged with during a medical procedure. The support sleeve 100 is configured to be positioned over the patient's arm. The support sleeve 100 may allow access to the patient's arm for introduction of a medical tool, facilitate movement of the arm between multiple positions, and/or stabilize the arm in a selected position while healthcare practitioner 2 conducts a medical procedure.

The support sleeve 100 includes a proximal end 101 that defines an arm opening 101a, a distal end 102, and a wall 103 that extends between the proximal end and the distal end. Support sleeve 100 may be positioned over an arm of patient 1 by positioning the arm through the arm opening at the proximal end 101. In use, the support sleeve 100 may stabilize the patient's arm in a pronated, palm-down position resting on the patient's torso. For example, the wrist may rest near the inguinal region. Interaction between the support sleeve 100, stabilization substrate 150, and/or the patient's body or another feature in the operating room at least partially maintains the patient's arm in the selected position. In some embodiments, the support sleeve 100 may at least partially maintain the patient's left arm in the pronated, palm down position while healthcare practitioner operates from the right side of the patient, or vice versa.

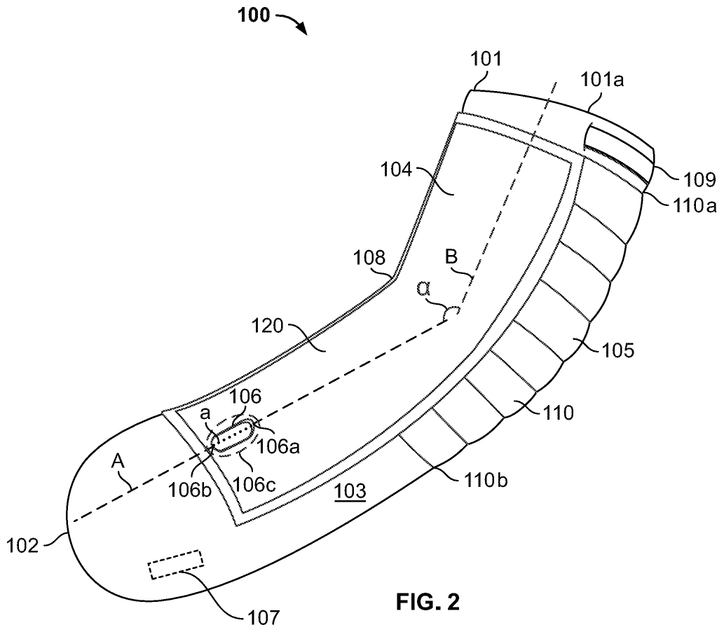

Referring to FIG. 2, a perspective view of example support sleeve 100 is shown. The support sleeve 100 includes a proximal end 101 that defines an arm opening 101a, and a distal end 102. The wall 103 extends between the proximal end 101 and the distal end 102. The wall includes an anterior side portion 104 and a posterior side portion 105. In some embodiments, the support sleeve 100 is configured to be positioned over a patient's arm such that the anterior side portion 104 of the wall 103 is located generally over the anterior side of the patient's arm, and the posterior side portion 105 of the wall 103 is located generally over the posterior side of the patient's arm.

The support sleeve 100 includes one or more access ports 106 that facilitate access of the patient's arm through the wall 103. For example, the access port 106 may define an opening through an entire thickness of the wall 103. A medical device may be passed through the access port 106 and into an access location through the patient's skin. In some embodiments, the access port 106 may include a removable liner that at least partially covers an opening defined by the access port 106. The removable liner may be removed at a time of use, such as in the operating room before or after the support sleeve 100 has been positioned over a patient's arm.

In an example embodiment, one or more of the access ports 106 are located to align with a patient's inner wrist when the support sleeve 100 is positioned on the patient's arm. For example, the access port 106 may be located on the anterior side portion 104 of the wall 103 proximate the distal end 102. In this way, a healthcare practitioner may access the radial artery or other vascular feature, for example, via the one or more access ports 106. Alternatively, or additionally, one or more access ports 106 may be located on the anterior side portion 104 of the wall 103 near the proximal end 101, near the inner elbow, etc. (e.g., to facilitate access of other locations on the anterior side of the patient's arm), and/or one or more access ports 106 may be located on the posterior side portion 105 of the wall 103 (e.g., to facilitate access of the posterior side of the patient's arm).

The support sleeve 100 may include one or more adhesive areas on an interior surface of the wall 103. For example, an adhesive 106c on an interior surface of the anterior side portion 104 at least partially surrounds the access port 106. Adhesive 106c promotes sterility by separating the access location of a patient's skin from the remainder of the patient's arm. In some embodiments, adhesive 106c further promotes stability by inhibiting relative movement between the support sleeve 100 and the patient's arm in use.

One or more access ports 106 may be located generally centrally on the anterior side portion 104 of the wall 103 proximate the distal end 102. For example, a central longitudinal axis (a) extending between a proximal end 106a and distal end 106b of the access port 106 may be collinear with a central longitudinal axis of the anterior side portion 104 of the wall 103. A generally centrally located access port 106 may facilitate alignment with the inner wrist surface when the support sleeve is positioned over the patient's arm.

Support sleeve 100 may include one or more features that promote patient comfort and/or protect the patient's arm. In an example embodiment, the support sleeve 100 includes padded area 110. Padded area 110 may include a compressible construction that protects and pads the patient's arm, such as padding configured to protect the patient's arm from compression neuropathy and decubitus wound injury, and for comfort. For example, padded area 110 may include locations of increased material thickness and/or may include one or more of foam, closed-cell foam, open-cell foam, compressible non-woven material, rubber, quilted material, laminated material, air pocket(s), water, gel, styrene bead, etc. In some embodiments, padded area 110 may at least partially be filled by an expandable material such that padded material can expand to at least partially conform to the patient's arm.

The padded area 110 may be present over some or all of the support sleeve 100. In an example embodiment, padded area 110 is located on the posterior side portion 105 of the wall 103. Padded area 110 may protect the elbow, triceps brachii, and other posterior features of the patient's arm, particularly when the patient's arm is resting/supported on the posterior side in a supinated position (e.g., during introduction of a medical device at the inner wrist), and/or while an arm is constrained by an arm board or other external structure in contact with the posterior side portion of the support sleeve 100. In an example embodiment, the padded area 110 extends from a proximal location 110a near proximal end 101 to a distal location 110b. Distal location 110b may be selected so that padded area 110 is present at the location of the patient's elbow (e.g., and extends just past the elbow location) when the support sleeve 100 is positioned on the patient's arm in use. Such a configuration may advantageously protect the patient's arm without inhibiting articulation of the support sleeve 100 or patient's arm (e.g., at an elbow location), and/or while facilitating access through access port 106.

The support sleeve 100 may include combinations of two or more materials that protect the patient's arm, and/or multiple padded areas 110 of different material construction. In an example embodiment, a shape-stable, compressible material 111 (FIG. 1) may be located at an elbow region. Compressible material 111 may provide additional padding, and/or support of a patient's elbow. In some embodiments, compressible material 111 may be a foam elbow pad (e.g., open-cell foam, closed-cell foam, etc.), that provides additional protection for the elbow and/or supports the elbow in an articulated position.

Support sleeve 100 is articulable at one or more locations between proximal end 101 and distal end 102. In an example embodiment, the support sleeve 100 includes a preformed articulated region 108. The articulated region 108 simulates an elbow bend and facilitates positioning of the patient's arm in a non-straight orientation. In some embodiments, the articulated region 108 provides a bend such that a lower or distal region of the support sleeve 100 has a first longitudinal axis (A) and the upper or proximal region of the support sleeve has a second longitudinal axis (B) angled relative to the first longitudinal axis (A) (e.g., when the support sleeve 100 is at rest not in use on a patient's arm). The articulated region 108 may thus provide a pre-defined bend that facilitates articulation and may reduce material bunching or gathering when the patient's arm is bent at the elbow while the support sleeve 100 is positioned on the user's arm. Alternatively, or additionally, articulated region 108 may facilitate intuitive positioning on a patient's arm because the support sleeve 100 is at least partially pre-formed in a natural, articulated positon, for example. In various example embodiments, the articulated region 108 includes a preformed bend that defines an angle between 10° and 90°, 30° and 60°, or about 45°. For example, an angle (a) defined between first longitudinal axis (A) and second longitudinal axis (B) may be between 10° and 90°, 30° and 60°, or about 45°. The articulated region 108 is flexible such that a patient's arm may be readily manipulated when the support sleeve 100 is positioned over the patient's arm. For example, the patient's arm can be manipulated during use such that angle (a) is increased or decreased.

The support sleeve 100 may have a tubular configuration such that the wall 103 defines a circular or rounded arm opening 101a and/or defines a circular or rounded cross-sectional space between the proximal end 101 and distal end 102. Such a configuration can facilitate positioning of the support sleeve 100 over a patient's arm, while providing an appropriate fit. In various example embodiments, the wall 103 may include a continuous material portion, such as a continuous material portion that is present at both the anterior side portion 104 and the posterior side portion 105. Ends of the continuous material portion may be joined (e.g., by stitching, welding, adhesive, etc.). In some embodiments, the support sleeve 100 may have few or no seams (e.g., such the support sleeve 100 has a seamless construction). Alternatively, or additionally, the wall 103 may include two or more planar sheets of material joined at longitudinal seams.

In various example embodiments, wall 103 may have a flexible, liquid-impermeable construction. For example, wall 103 may include one or more layers of polyester, polyethylene, cotton, paper, non-woven, fabric, etc. Alternatively, or additionally, wall 103 may include one of more layers that impart liquid-impermeability, such as a polymer layer. In some embodiments, wall 103 may have a laminated construction of two or more layers. The layers may be microporous to promote breathability while being liquid-impermeable. In some embodiments, the support sleeve 100 may be a stretchable material, such that the wall 103 may stretch and flex to accommodate the patient's arm, while being in close contact and or slight compression against the patient's arm in at least some locations. For example, in use, the wall 103 may be slightly stretched in at least some locations when positioned on the patient's arm. The support sleeve 100 may be a sterile support sleeve that protects the patient from skin flora and the external environment.

The support sleeve 100 may have one or more securing features that promote a secure fit on the patient's arm, and/or inhibit relative movement between the support sleeve 100 and the patient's arm during use (e.g., relative rotation and/or longitudinal translation). For example, the support sleeve may include one or more securing features 109 near proximal end 101. Securing features 109 may include one or more of an elasticized region, strap, tie, hook-and-loop fastener, adjustment mechanism, etc. that promotes a secure fit around the patient's upper arm. In an example embodiment, the securing feature 109 does not extend around an entire perimeter of the wall 103. One or more securing features 109 may be located elsewhere between proximal end 101 and distal end 102, such as above the articulated region 108, below the articulated region 108, proximate the access port 106, etc.

In various example embodiments, the support sleeve 100 may include one or more finger and/or thumb portions configured to receive an individual finger/thumb. Such a configuration may facilitate positioning the support sleeve, and/or promote patient comfort. In an example embodiment, the support sleeve includes a pulse oximeter 107 in the sleeve configured to monitor (e.g., continuously) the patient's hand oxygen supply.

The support sleeve 100 may be configured to at least partially stabilize a patient's arm in a selected position. In an example embodiment, the support sleeve 100 includes one or more attachment devices 120 that facilitate engagement with a substrate, such as a complementary stabilization substrate in the operating environment. For example, attachment device 120 includes an area that, when brought into contact with another surface, engages the surface to limit relative movement between the support sleeve 100 and the surface. In various example embodiments, the attachment device 120 includes one or more of adhesive, hook-and-loop fastener, self-adherent non-woven material, self-adherent polymer material, highly frictional polymer material, coadhesive material, etc. In some embodiments, the attachment device 120 may be exposed (e.g., by removing a liner covering an adhesive), and the attachment device 120 brought into contact with a stabilization substrate, such as a patient's skin, sterile surgical drape, belt, neckband, strap, other stabilization substrate, etc.

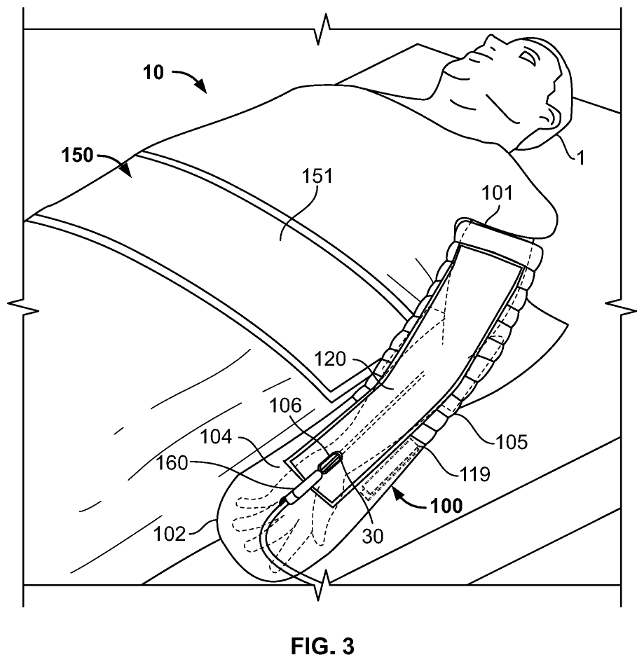

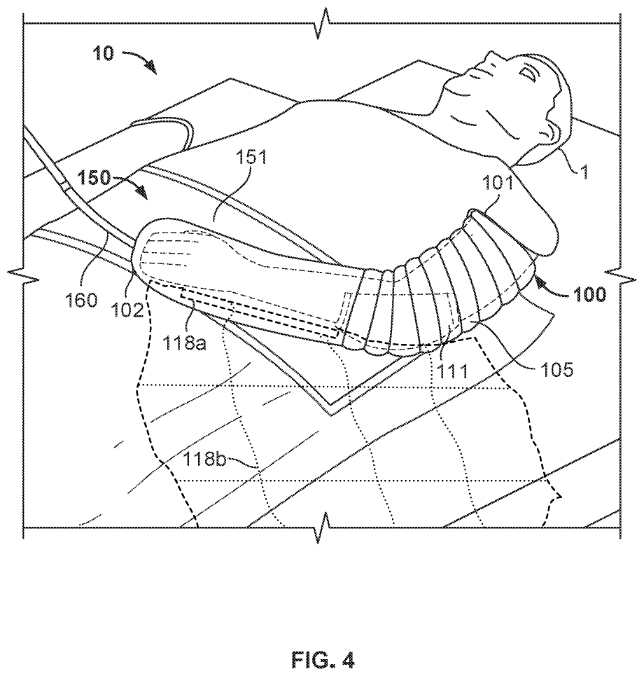

Referring to FIGS. 3 and 4, the example support sleeve 100 is shown in use in a first position (FIG. 3) and a second position (FIG. 4). In the first position, the arm is in a supinated position in which the anterior side of the patient's arm is facing upwards. The inner-wrist is thus facing upwards and accessible (e.g., via access port 106) by a healthcare practitioner. An medical device may thus be introduced into the patient's arm proximate the inner wrist, such as to access the radial artery or other feature of the patient.

In various example embodiments, medical device 160 may include a catheter, such as an access sheath, vascular sheath, endovascular medical device, or the like. The medical device may be configured to extend through a perforation in patient 1 at access location 30 and into an anatomical conduit, such as the radial artery, other blood vessel, etc. In an example embodiment, the catheter of the medical device includes a proximal end external of patient 1, a distal end internal to patient 1, and defines a lumen extending at least partially between the proximal and distal ends. The lumen defines a pathway for delivery or withdrawal of fluids, instruments such as guide wires or other interventional tools, or a combination thereof. The catheter may thus accommodate the manipulation and passage of one or more guide wires or interventional tools while the catheter remains in a fixed position relative to access location 30.

The medical device 160 may be effectively introduced at access location 30 while the patient's arm is in the first position within the support sleeve 100. A supinated position may promote access of the radial artery of other anatomical conduit from the anterior side of the patient's arm while the arm rests/is supported on the posterior side of the arm.

One or more attachment devices 120 are located on at least a portion of the anterior side portion 104 of the support sleeve 100. In an example embodiment, one or more attachment devices 120 are positioned both distally and proximally of the access port 106, and/or may at least partially surround the access port 106. For example, the attachment device 120 may include an area that extends from a location distal of the access port 106 to a location near the proximal end 101 of the support sleeve 100. Alternatively, or additionally, the attachment device 120 may be present only on the anterior side portion 104 of wall 103 (e.g., not present on the posterior side portion 105 of wall 103). In other example embodiments, attachment device may be present only on the posterior side portion 105 or both the anterior side portion 104 and the posterior side portion 105. In some example embodiments, one or more attachment devices 120, such as a strap, may extend at least partially around support sleeve 100 and promote pronation, or supination, when attached to a substrate.

The medical sleeve system 10 includes a stabilization substrate 150 that the support sleeve 100 may be engaged with to at least partially support the patient's arm in a selected position. In an example embodiment, the stabilization substrate 150 may be a surgical drape or pad positionable over at least a portion of the patient's torso, and/or at least partially behind the patient, attached to the operating table, other structure in the operating room, etc. The stabilization substrate 150 may include material complementary to the support sleeve 100 such that the support sleeve may be at least partially maintained in engagement with the stabilization substrate 150. For example, the stabilization substrate 150 may include a complementary attachment device 151 that includes one or more of an adhesive, hook-and-loop fastener, self-adherent material, highly frictional polymer, etc., complementary to attachment device 120, wall 103, and/or other feature of the support sleeve 100.

In some embodiments, both the stabilization substrate 150 and the support sleeve 100 include attachment devices, and the attachment devices are complementary to one another. For example, the support sleeve 100 includes a hook-and-loop attachment device 120 (e.g., the hook side of the hook-and-loop fastener) and the stabilization substrate 150 includes a complementary hook-and-loop attachment device (e.g., the loop side of the hook-and-loop fastener). The hook and loop sides of the support sleeve 100 and stabilization substrate 150 may be brought into engagement to inhibit relative movement and at least partially support the support sleeve 100 in a selected position. Alternatively, or additionally, the support sleeve 100 may include an adhesive attachment device 120 and the stabilization substrate 150 includes a complementary adhesive attachment device or a material conducive to engagement with the adhesive of the support sleeve 100. The adhesive attachment device 120 of the support sleeve 100 may be brought into engagement with the stabilization substrate 150 to inhibit relative movement and at least partially support the support sleeve in a selected position.

In some embodiments, only one of the support sleeve 100 or stabilization substrate 150 include an attachment device. For example, the support sleeve 100 may include an adhesive attachment device 120 engageable with a surface of the stabilization substrate 150, and the stabilization substrate 150 does not include an additional adhesive attachment device, or vice versa.

The support sleeve 100, and patient's arm within the support sleeve 100, may be manipulated into a selected position by moving from a first position (FIG. 3) to a second position (FIG. 4). The support sleeve 100 at least partially maintains the patient's arm in the selected position, such as by engagement with stabilization substrate 150. For example, the patient's arm can be manipulated between the first position and the second position while the support sleeve 100 is located on the patient's arm and the medical device 160 is fixedly positioned through the access location 30. After the medical device 160 has been introduced, the arm may be rotated medially from the first, supinated position, to a second, pronated position, in which the arm rests in a palm-down position at least partially on the patient's torso. Alternatively, or additionally, the angle of bend at the patient's elbow, and/or other parameters of the patient's arm orientation, may be adjusted to bring the patient's arm into a selected position. In some embodiments, the patient's arm is oriented such that the inner wrist is located near the inguinal region.

Engagement between the support sleeve 100 and the stabilization substrate 150 inhibits relative movement between the support sleeve 100 and the stabilization substrate 150, and promotes support of the patient's arm in the selected position. For example, the healthcare practitioner may simply lift the patient's arm, move into the selected position, and bring into contact with the stabilization substrate 150. The medical sleeve system 10 thus promotes efficient set-up and support of the patient's arm in the selected position. Moreover, in some embodiments, the support sleeve 100 may be repositionable relative to the stabilization substrate 150, such that the healthcare practitioner may readily separate the support sleeve 100 from the stabilization substrate 150 to make minor adjustments to the arm position, monitor the access location 30 or other portions of the anterior side of the arm, etc.

In the example second position shown in FIG. 4, the patient's arm is in a natural, palm-down position in which the arm rests on the torso with the wrist near the inguinal region. Such a position may be analogous to a natural sleeping position and promote patient comfort. Alternatively, or additionally, the second position may promote ergonomic manipulation of the medical device 160 by the healthcare practitioner, such as by allowing the healthcare practitioner to operate across the patient's body, primarily with a dominant operating hand, and/or further from a radiation field employed during the medical procedure, etc. In some example embodiments, an additional support structure may be positioned along at least a portion of the patient's arm, such as along a posterior side of the patient's arm (e.g., along the triceps brachii). The support structure may be a rigid arm board or other support structure that further supports the arm in the second position and/or prevents movement of the arm away from the body. The padded areas 110 (e.g., and elbow pad 111) may protect the patient's arm and promote patient comfort when supported by an additional structure.

The support sleeve 100 facilitates positioning of the patient's arm in one or more selected positions based on the healthcare practitioner's preferences, patient anatomy, the medical procedure being performed, etc. The second position is not limited to an orientation in which the elbow is bent at about 90°. The healthcare may select an appropriate angle, including angles that allow the wrist to rest at a location near the inguinal region. The material and construction of the support sleeve 100 allow articulation of the patient's arm between multiple positions, while supporting the patient's arm in the selected position.

In various example embodiments, the support sleeve 100 may include one or more expandable portions, such as an expandable sterile drape 118. During use, the expandable sterile drape 118 may be expanded (e.g., unfolded, unrolled, untucked, etc.) to cover selected portions of the patient's arm, body, and/or the operating table, other surfaces, etc. The expandable sterile drape 118 may be manipulated between a folded or unexpanded configuration 118a, and expanded configuration. In an example embodiment, the expandable sterile drape is folded, rolled, tucked, etc. at a location of the support sleeve 100 proximate an ulnar region of the patient's arm (e.g., between the anterior and posterior wall portions 104, 105). The expandable sterile drape 118 may be unexpanded while the patient's arm is manipulated into the supinated position. When the patient's arm is in the supinated position (e.g., after the medical device has been introduced and the arm), the expandable sterile drape 118 may be accessible such that the healthcare practitioner may expand the sterile drape 118 and position in a selected location. In some embodiments, expandable sterile drape 118 may promote operating field sterility by covering one or more locations where the patient's arm was positioned during introduction of a medical device or other access to the patient's arm.

Expandable sterile drape 118 may be formed as a single unitary component with one or more other portions of support sleeve 100. For example, expandable sterile drape 118 may formed as a portion of the support sleeve 100, such as the wall 103. Alternatively, or additionally, expandable sterile drape 118 may be formed separately and subsequently attached to one or more portions of support sleeve 100, such as the wall 103. The expandable sterile drape 118 may include one or more fold lines 118b. A healthcare practitioner may expand the sterile drape 118 to a selected degree by unfolding the sterile drape 118 at one or more fold lines 118b.

Expandable sterile drape 118 may include one or more radiation-blocking materials to shield radiation from the patient and/or healthcare practitioner. In an example embodiment, expandable sterile drape 118 is positioned over at least a portion of the posterior wall portion 105 the support sleeve 100 (e.g., such that the posterior wall portion 105 is between the expandable sterile drape 118 and the patient's arm).

Alternatively, or additionally, the support sleeve may include one or more expandable portions proximate the anterior side portion of the support sleeve 100. For example, the support sleeve 100 may include an expandable sterile drape 119 (FIG. 3) that may be manipulated between a folded or unexpanded configuration and an expanded configuration. The expandable sterile drape may be positioned under at least a portion of the anterior wall portion 104 of the support sleeve 100 (e.g., such that the anterior wall portion 104 is between the expandable sterile drape 119 and the patient's arm), and may cover the access port 106. The expandable sterile drape 119 may include one or more radiation-blocking materials to provide radiation shielding for the patient and/or healthcare practitioner.

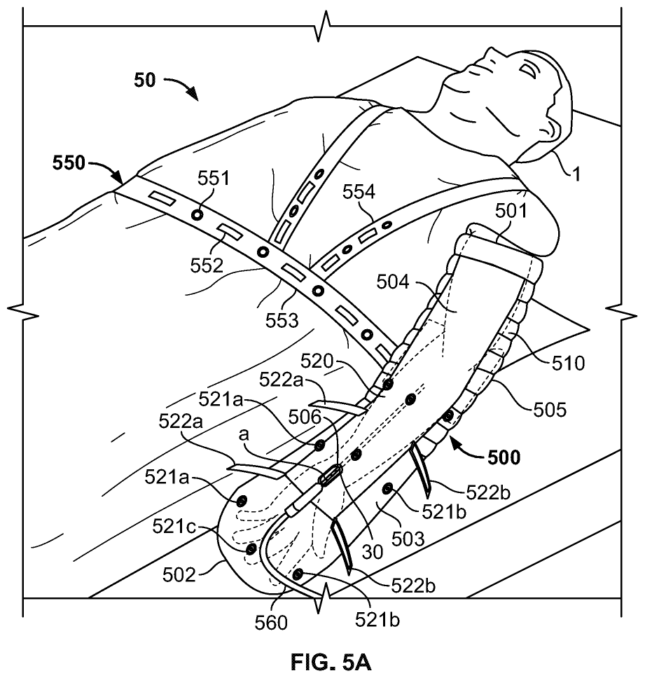

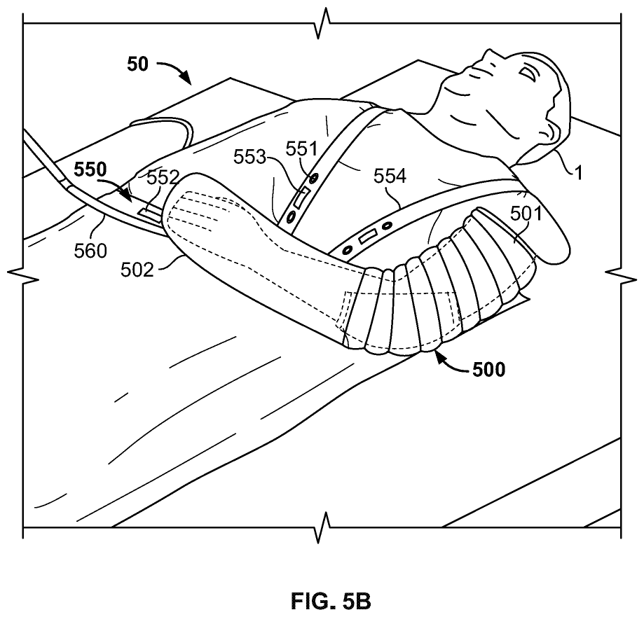

Referring now to FIGS. 5A-5B, an example medical sleeve system 50 is shown, including support sleeve 500, stabilization substrate 550. The support sleeve 500 is configured to be positioned over the patient's arm. The support sleeve 500 may allow access to the patient's arm for introduction of a medical tool, facilitate movement of the arm between multiple positions, and/or stabilize the arm in a selected position while a healthcare practitioner conducts a medical procedure. In various example embodiments, medical sleeve system 50 may include one or more features as described herein with reference to medical sleeve system 10 described with reference to FIGS. 1-4.

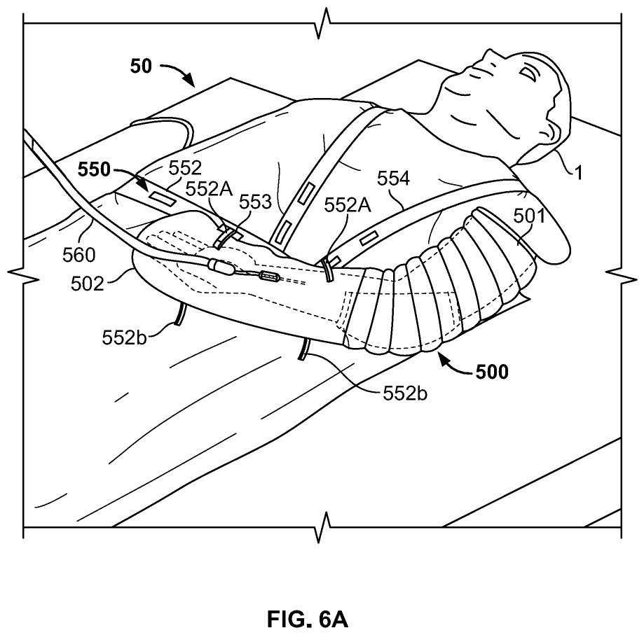

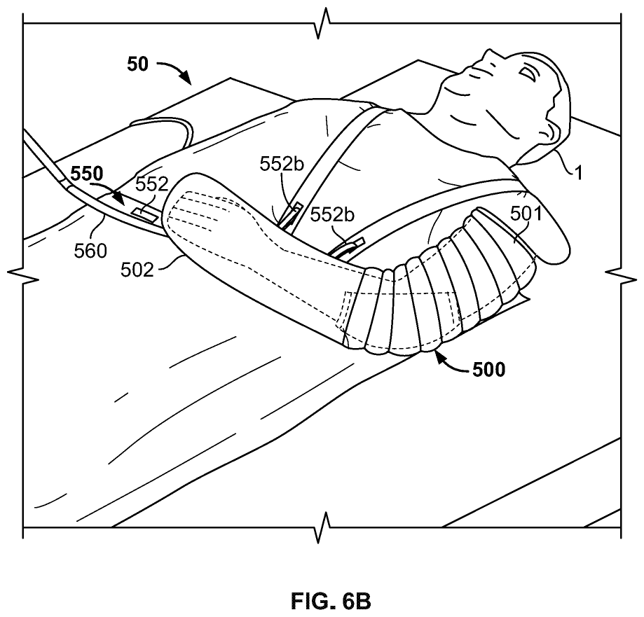

The support sleeve 500 includes a proximal end 501 that defines an arm opening, a distal end 502, and a wall 503 that extends between the proximal end 501 and the distal end 502. Support sleeve 500 may be positioned over an arm of patient 1 by positioning the arm through the arm opening at the proximal end 501. In use, the support sleeve 500 may stabilize the patient's arm in a pronated, palm-down position resting on the patient's torso (FIG. 6). For example, the wrist may rest near the inguinal region. Interaction between the support sleeve 500, stabilization substrate 550, and/or the patient's body or another feature in the operating room at least partially maintains the patient's arm in the selected position. In some embodiments, the support sleeve 500 may at least partially maintain the patient's left arm in the pronated, palm down position while healthcare practitioner operates from the right side of the patient, or vice versa.

The wall 503 of support sleeve 500 includes an anterior side portion 504 and a posterior side portion 505. In some embodiments, the support sleeve 500 is configured to be positioned over a patient's arm such that the anterior side portion 504 of the wall 503 is located generally over the anterior side of the patient's arm, and the posterior side portion 505 of the wall 503 is located generally over the posterior side of the patient's arm. The support sleeve 500 includes one or more access ports 506 that facilitate access of the patient's arm through the wall 503. In some embodiments, the access port 506 may include a removable liner that at least partially covers an opening defined by the access port 506. The removable liner may be removed at a time of use, such as in the operating room before or after the support sleeve 500 has been positioned over a patient's arm.

One or more of the access ports 506 may be configured for alignment with a patient's inner wrist when the support sleeve 500 is positioned on the patient's arm. For example, the access port 506 may be located on the anterior side portion 504 of the wall 503 proximate the distal end 502. In this way, a healthcare practitioner may access the radial artery or other vascular feature, for example, via the one or more access ports 506. Alternatively, or additionally, one or more access ports 506 may be located on the anterior side portion 504 of the wall 503 near the proximal end 501, near the inner elbow, etc. (e.g., to facilitate access of other locations on the anterior side of the patient's arm), and/or one or more access ports 506 may be located on the posterior side portion 505 of the wall 503 (e.g., to facilitate access of the posterior side of the patient's arm).

One or more access ports 506 may be located generally centrally on the anterior side portion 504 of the wall 503 proximate the distal end 502. For example, a central longitudinal axis (a) extending between a proximal end and distal end of the access port 506 may be collinear with a central longitudinal axis of the anterior side portion 504 of the wall 503. A generally centrally located access port 506 may facilitate alignment with the inner wrist surface when the support sleeve is positioned over the patient's arm.

Support sleeve 500 may include one or more features that promote patient comfort and/or protect the patient's arm. In an example embodiment, the support sleeve 500 includes padded area 510. Padded area 510 may include a compressible construction that protects and pads the patient's arm. For example, padded area 510 may include locations of increased material thickness and/or may include one or more of foam, closed-cell foam, open-cell foam, compressible non-woven material, rubber, quilted material, laminated material, air pocket(s), gel, etc. In some embodiments, padded area 510 may at least partially be filled by an expandable material such that padded material can expand to at least partially conform to the patient's arm.

The padded area 510 may be present over some or all of the support sleeve 500. In an example embodiment, padded area 510 is located on the posterior side portion 505 of the wall 503. In an example embodiment, the padded area 510 extends from a proximal location near proximal end 501 to a distal location. The distal location may be selected so that padded area 510 is present at the location of the patient's elbow (e.g., and extends just past the elbow location) when the support sleeve 500 is positioned on the patient's arm in use. Such a configuration may advantageously protect the patient's arm without inhibiting articulation of the support sleeve 500 or patient's arm (e.g., at an elbow location), and/or while facilitating access through access port 506.

In various example embodiments, wall 503 may have a flexible, liquid-impermeable construction. For example, wall 503 may include one or more layers of polyester, polyethylene, cotton, paper, non-woven, fabric, etc. Alternatively, or additionally, wall 503 may include one of more layers that impart liquid-impermeability, such as a polymer layer. In some embodiments, wall 503 may have a laminated construction of two or more layers. The layers may be microporous to promote breathability while being liquid-impermeable. In some embodiments, the support sleeve 500 may be a stretchable material, such that the wall 503 may stretch and flex to accommodate the patient's arm, while being in close contact and or slight compression against the patient's arm in at least some locations. For example, in use, the wall 503 may be slightly stretched in at least some locations when positioned on the patient's arm. The support sleeve 500 may be a sterile support sleeve that protects the patient from skin flora and the external environment.

The medical sleeve system 50 may be configured to at least partially stabilize a patient's arm in a selected position. In an example embodiment, the support sleeve 500 includes one or more attachment devices 520 that facilitate engagement with a substrate, such as a complementary stabilization substrate 550 in the operating environment. For example, attachment devices 520 are engageable with complementary features of stabilization substrate 550 to limit relative movement between the support sleeve 500 and the stabilization substrate 550.

The example support sleeve 500 is shown in use in a first position (FIG. 5A) and a second position (FIG. 5B). In the first position, the arm is in a supinated position in which the anterior side of the patient's arm is facing upwards. The inner-wrist is thus facing upwards and accessible (e.g., via access port 506) by a healthcare practitioner. A medical device 560 may thus be introduced into the patient's arm proximate the inner wrist, such as to access the radial artery or other feature of the patient. The medical device 560 may be effectively introduced at access location 530 while the patient's arm is in the first position within the support sleeve 500. A supinated position may promote access of the radial artery of other anatomical conduit from the anterior side of the patient's arm while the arm rests/is supported on the posterior side of the arm.

In various example embodiments, the support sleeve 500 includes one or more attachment devices 520, such as one or more of adhesive straps, hook-and-loop straps, clips, hooks, snaps, ties, loops, etc., that are engageable with a complementary feature of stabilization substrate 550. The stabilization substrate 550 may likewise include one or more complementary attachment devices, such as adhesive straps hook-and-loop straps, clips, hooks, snaps, ties, loops, etc. For example, the attachment devices 520 of the support sleeve 500 may include one or more snaps 521 engageable with complementary snaps 551 of stabilization substrate 550. Snaps 521 are fixedly positioned relative to one another along wall 503. In an example embodiment, one or more snaps 521c are located centrally on the anterior side portion 504, such as along a central longitudinal axis of the support sleeve 500. Alternatively, or additionally, one or more snaps 521 are not-centrally located, offset from the central longitudinal axis (e.g., offset from longitudinal axis (a) of the access port 506. For example, snaps 521a are located near an inner or bottom side of the support sleeve 500 (e.g., a side proximate a patient's little finger or digitus minimus manus), and snaps 521b are located near an outer or top side of the support sleeve (e.g., a side proximate a patient's thumb). Multiple snaps 521 at varied locations of the support sleeve 500 facilitate positioning of the patient's arm in a selected position.

Alternatively, or additionally, attachment devices 520 of the support sleeve 500 may include one or more straps 522. The straps 522 may be configured to attach to a complementary strap, loop, etc., of the stabilization substrate. Multiple straps 522 may be positioned on wall 503 of the support sleeve 500 and facilitate engagement between the support sleeve 500 and the stabilization substrate in a selected position. In some embodiments, straps 522a, 522b, are offset from the central longitudinal axis. Offset straps 552 may facilitate manipulation of a patient's arm while the support sleeve 500 remains engaged with the stabilization substrate 550, such that the patient's arm may be rotated slightly from a pronated position towards a supinated position while one or more straps located near a bottom edge of the support sleeve 500 (e.g., straps 522a), for example, are engaged with the stabilization substrate 550. Likewise, straps 522b may limit or prevent rotation when attached.

Stabilization substrate 550 may include one or more straps, such as a belt 553 and/or neck or shoulder straps 554, that secure the stabilization substrate 550 relative to the patient 1. Belt 553 may pass at least partially behind patient 1 (e.g., around the patient's waist, torso, chest, etc.). Straps 554 may pass around the patient's neck or may pass over the shoulders (e.g., and extend under the patient and connect to belt 553 on the posterior side of the patient. In various example embodiments, stabilization substrate 550 may include only belt 553, only one or more straps 554, and/or a combination of belt 553 and straps 554. The belt 553 and/or straps 554 may be integral or otherwise connected with a surgical drape that at least partially covers the patient. Alternatively, the belt and/or straps 553, 554 may be separate from a surgical drape (e.g., separately positioned over the surgical drape).

The support sleeve 500, and patient's arm within the support sleeve 500, may be manipulated into a selected position by moving from a first position (FIG. 5A) to a second position (FIG. 5B). The support sleeve 500 at least partially maintains the patient's arm in the selected position, such as by engagement with stabilization substrate 550. For example, the patient's arm can be manipulated between the first position and the second position while the support sleeve 500 is located on the patient's arm and the medical device 560 is fixedly positioned through the access location 530. After the medical device 560 has been introduced, the arm may be rotated medially from the first, supinated position, to a second, pronated position, in which the arm rests in a palm-down position at least partially on the patient's torso. Alternatively, or additionally, the angle of bend at the patient's elbow, and/or other parameters of the patient's arm orientation, may be adjusted to bring the patient's arm into a selected position. In some embodiments, the patient's arm is oriented such that the inner wrist is located near the inguinal region.

Engagement between the support sleeve 500 and the stabilization substrate 550 inhibits relative movement between the support sleeve 500 and the stabilization substrate 550, and promotes support of the patient's arm in the selected position. For example, the healthcare practitioner may simply lift the patient's arm, move into the selected position, and engage one or more attachment devices 520 of the support sleeve 500 and/or attachment devices 551 of the stabilization substrate 550. The presence of multiple attachment devices of the stabilization substrate 550 and/or the support sleeve 500 facilitates engagement in the selected position. The medical sleeve system 50 may promote efficient set-up and support of the patient's arm in the selected position. Moreover, in some embodiments, the support sleeve 500 may be repositionable relative to the stabilization substrate 550 (e.g., by disengaging one or more of the attachment devices) such that the healthcare practitioner may readily separate the support sleeve 500 from the stabilization substrate 550 to make minor adjustments to the arm position, monitor the access location 530 or other portions of the anterior side of the arm, etc.

The support sleeve 500 facilitates positioning of the patient's arm in one or more selected positions based on the healthcare practitioner's preferences, patient anatomy, the medical procedure being performed, etc. The second position is not limited to an orientation in which the elbow is bent at about 90°. The healthcare may select an appropriate angle, including angles that allow the wrist to rest at a location near the inguinal region. The material and construction of the support sleeve 500 allow articulation of the patient's arm between multiple positions, while supporting the patient's arm in the selected position.

In the example second position shown in FIG. 5B, the patient's arm is in a natural, palm-down position in which the arm rests on the torso with the wrist near the inguinal region. Such a position may be analogous to a natural sleeping position and promote patient comfort. Alternatively, or additionally, the second position may promote ergonomic manipulation of the medical device 560 by the healthcare practitioner, such as by allowing the healthcare practitioner to operate across the patient's body, primarily with a dominant operating hand, and/or further from a radiation field employed during the medical procedure, etc. In some example embodiments, an additional support structure may be positioned along at least a portion of the patient's arm, such as along a posterior side of the patient's arm (e.g., along the triceps brachii). The support structure may be a rigid arm board or other support structure that further supports the arm in the second position and/or prevents movement of the arm away from the body. The padded areas 510 may protect the patient's arm and promote patient comfort when supported by an additional structure.

In some embodiments, snaps 521 and/or straps 522 that are offset from the central longitudinal axis facilitate manipulation of a patient's arm while the support sleeve 500 remains engaged with the stabilization substrate 550. For example, a patient's arm may be rotated slightly from a pronated position towards a supinated position while one or more snaps or straps 521a, 522a located near a bottom side of the support sleeve 500 (e.g., a side proximate a patient's little finger or digitus minimus manus) are engaged with the stabilization substrate 550. In this way, a healthcare practitioner may monitor access location 30, and or other features on the anterior side of the patient's arm, without fully disengaging the support sleeve 500 from the stabilization substrate 550. The snaps 521a and/or straps 522a may function similar to a hinge such that the patient's arm is maintained in a position across the torso while allowed to rotate. One or more snaps 521, straps 522, etc. located near a top side or a patient's thumb can be secured to prevent or limit rotation towards a supinated position, for example by attachment to straps 552 and/or 554.

For example, medical sleeve system 50 is shown in FIGS. 6A and 6B with snaps 521 omitted for illustrative purposes. FIG. 6A shows the patient's arm in a supinated position in which support sleeve 500 is attached to stabilization substrate 550. Straps 552a are engaged with strap 552 such that the support sleeve 500, and the patient's arm within the support sleeve 500, is rotatable about the attachment location. In this way, a physician may rotate the patient's arm from a partially supinated position (FIG. 6A) to the pronated position (FIG. 6B), or vice versa, while the support sleeve 500 is attached to stabilization substrate 550. When in the pronated position, attachment of straps 522b to the stabilization substrate 550, such as to straps 554, may prevent or limit rotation towards the supinated position of FIG. 6A.

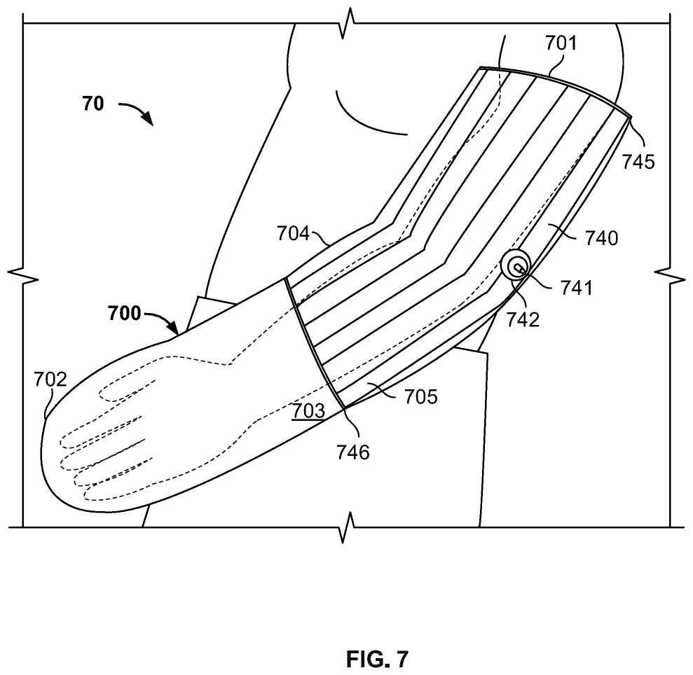

Referring now to FIG. 7, an example medical sleeve system 70 is shown, including support sleeve 700 having an expandable portion 740. The support sleeve 700 is configured to be positioned over the patient's 1 arm. The support sleeve 700 may allow access to the patient's arm for introduction of a medical tool, facilitate movement of the arm between multiple positions, and/or stabilize the arm in a selected position while healthcare practitioner 2 conducts a medical procedure. In various example embodiments, medical sleeve system 70 may include one or more features as described herein with reference to medical sleeve systems 10, and 50, described with reference to FIGS. 1-6.

The support sleeve 700 includes a proximal end 701 that defines an arm opening, a distal end 702, and a wall 703 that extends between the proximal end 701 and the distal end 702. Support sleeve 700 may be positioned over an arm of patient 1 by positioning the arm through the arm opening at the proximal end 701.

The support sleeve 700 includes an expandable portion 740. The expandable portion includes one or more regions that can change volume. For example, after positioning the support sleeve on the patient's arm, expandable portion 740 may be expanded to conform and/or slightly compress around the patient's arm. The expandable portion 740 may provide additional support for the patient's arm, promote a particular angle/orientation, inhibit relative movement between the patient's arm and the support sleeve 700, and/or provide additional protection for the patient's arm.

In an example embodiment, expandable portion 740 includes one or more inflatable air bladders that are expandable via pressurized air. For example, the expandable portion may include an inlet 741 that can be connected to a pressurized air source, such as an air compressor, air canister, manual pump, etc., to expand the expandable portion 740. Alternatively, or additionally, expandable portion 740 may include an integrated manual pump 742. The manual pump 742 can be squeezed or otherwise manipulated to draw air through the inlet to expand the expandable portion 740.

In various other example embodiments, expandable portion 740 may include an expandable material, such as an expandable foam, phase-change material, thermally-activated expandable material, etc. The material may irreversibly expandable. An irreversibly expandable material may prevent reuse and promote sterility.

The expandable portion 740 may be located over only a portion of the support sleeve 700, and not present throughout the entire support sleeve 700. In an example embodiment, the expandable portion 740 extends from a proximal end 745 near proximal end 701 of the support sleeve 700 to a distal end 746 located distally of the elbow (e.g., but does not extend to the distal end 702 of the support sleeve 700). Alternatively, or additionally, the expandable portion 740 may extend around the entire perimeter of the support sleeve 700, or may extend around less than the entire perimeter of the support sleeve 700. In an example embodiment, the expandable portion 740 covers the entire posterior side portion 705 of the support sleeve 700 between the proximal end 745 and the distal end 746 of the expandable portion 740. For example, that expandable portion may not be present on at least a portion of the anterior side portion 704 along the entire length between the proximal end 701 and the distal end 702.

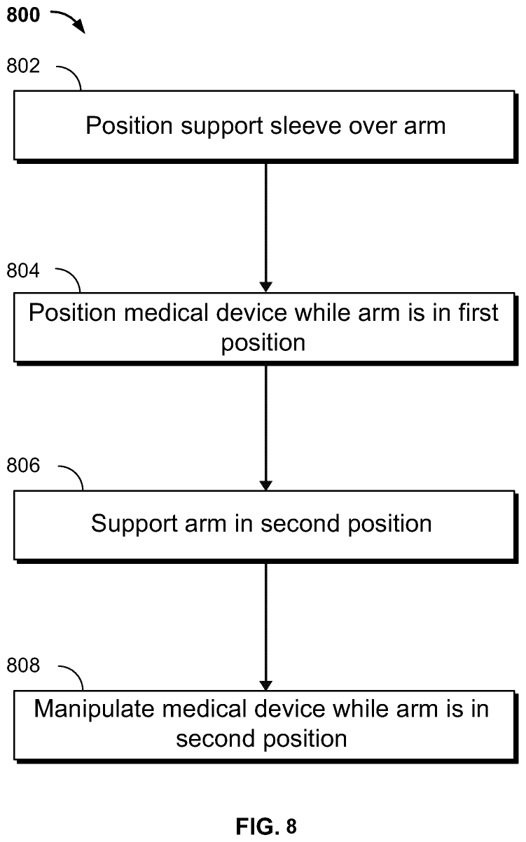

Referring to FIG. 8, an example flow diagram is shown, illustrating a method of performing a medical procedure using an example medical sleeve system. Example method 800 may include operation 802 of positioning a support sleeve over a patient's arm. The support sleeve may be positioned such that an access port of the support sleeve is aligned with an inner wrist of the patient. In some embodiments, before the support sleeve is positioned over the patient's arm, the patient's arm is sterilized, particularly the access location of the patient's skin, such as the inner wrist.

Example method 800 may include operation 804 of positioning a medical device through an access point in an anatomical vessel of a patient via an access port of the support sleeve while the patient's arm is in a first position. The medical device may be a catheter such as an access sheath, vascular sheath, endovascular medical device, or the like, configured to extend through a perforation in a patient at the access point and into an anatomical conduit, such as the radial artery or other blood vessel. In an example embodiment, after insertion, the catheter has a proximal end external to the patient, a distal end internal to the patient, and at least one lumen extending between the proximal and distal ends. The lumen defines a pathway for delivery or withdrawal of fluids, instruments such as guide wires or other interventional tools, or a combination thereof.

In an example embodiment, the first position may be a supinated position in which the patient's palm and inner wrist face upwardly while the arm is at, or extending from, the patient's side.

Example method 800 may include operation 806 of supporting the patient's arm in a second, selected position that is different from the first position. For example, the healthcare practitioner may manipulate the patient's arm into a selected position, and may support the patient's arm in the selected position by engaging one or more attachment features of the medical sleeve system. The patient's arm may be supported by engagement between one or more attachment devices of the support sleeve with a stabilization substrate, such as one or more of adhesive, hook-and-loop fastener, self-adherent non-woven, self-adherent polymer, highly frictional polymer, snaps, straps, ties, frictional materials, coadhesive materials, etc.

Example method 800 may include operation 808 carrying out one or more operations of a medical procedure while the patient's arm is supported in the second position. The medical procedure may involve radial access (e.g., such as left radial access by a practitioner operating from the right side of the patient) and may include coronary catheterization, percutaneous peripheral intervention, percutaneous coronary intervention, carotid artery and subclavian artery intervention for acute strokes, treatment of vascular malformations, and aneurysms, central vein access for electrophysiology procedures, hemodialysis intervention, arteriovenous fistula (AVF) intervention, electrophysiological cardiology procedures, pacemaker insertions, mesenteric and renal arterial system, and/or other applications.

Example method 800 may optionally include one or more additional operations, such as removing the support sleeve from the patient, and safely disposing of the support sleeve (e.g., disposing with other surgical draping used during the medical procedure).

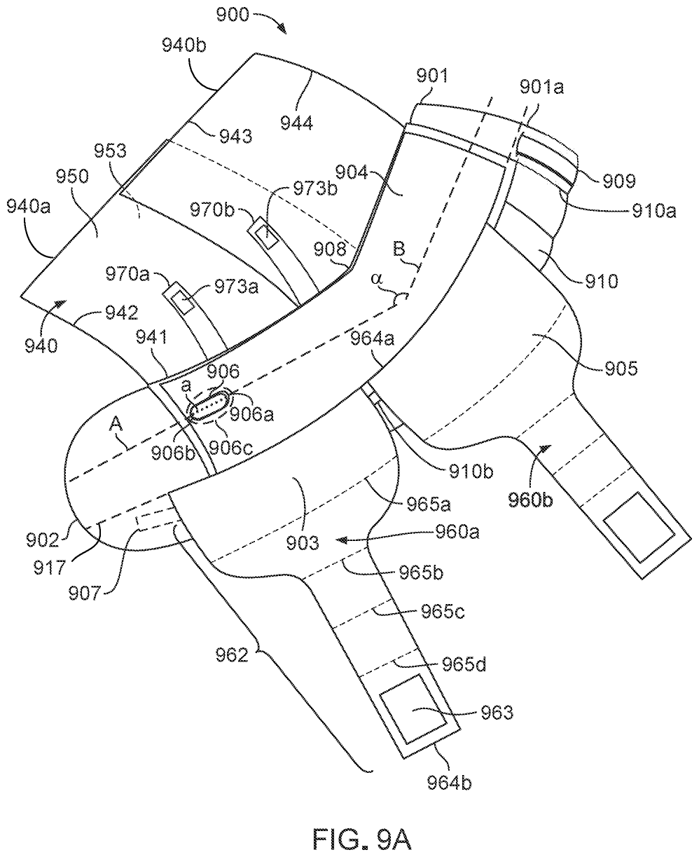

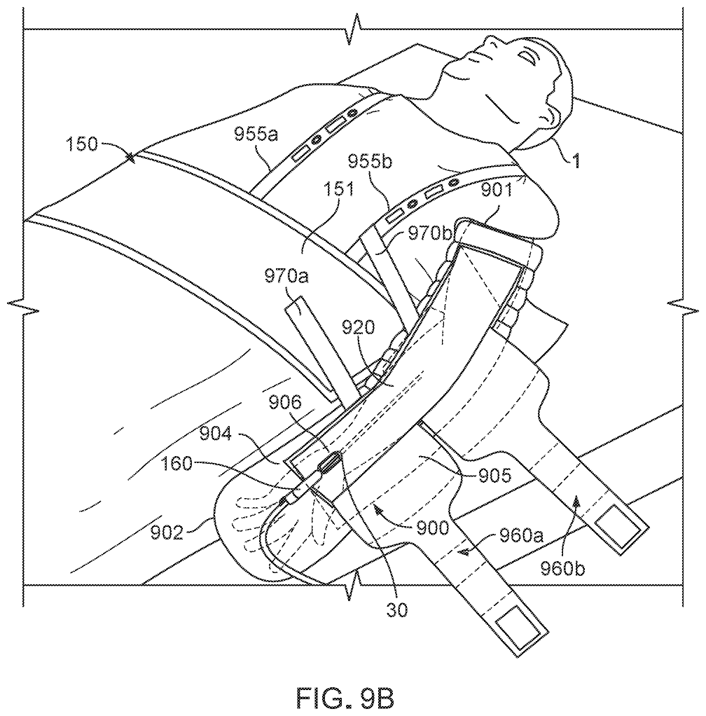

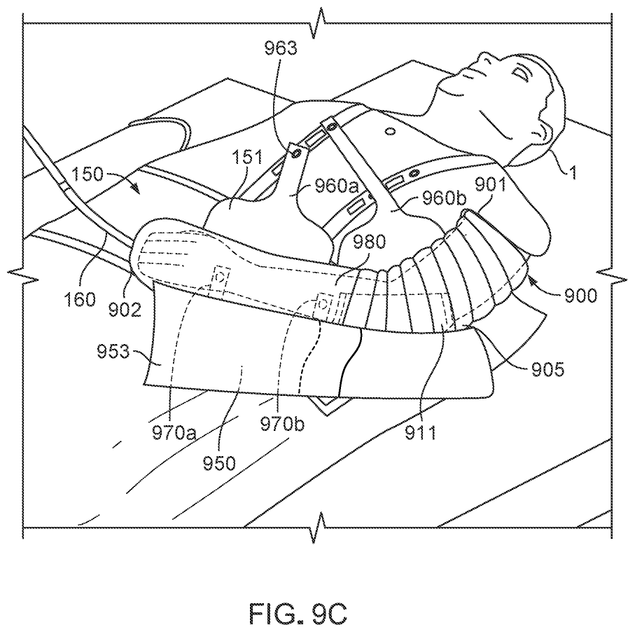

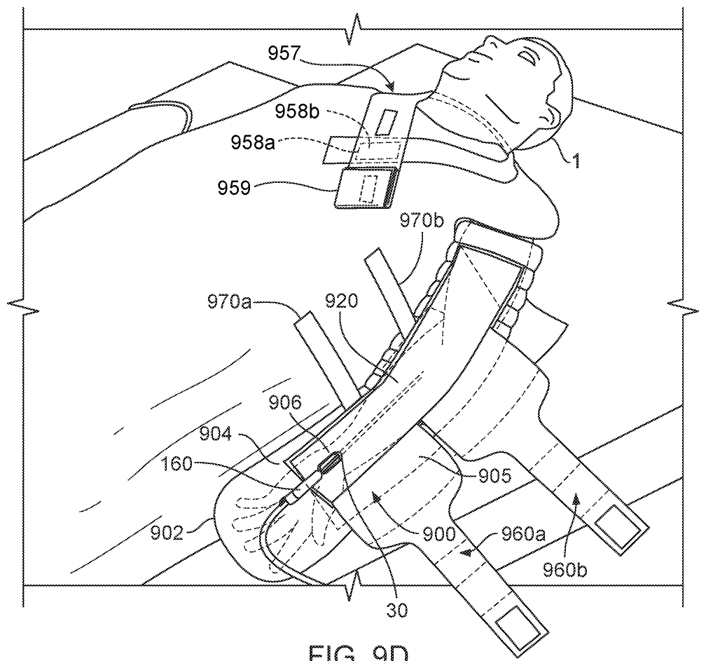

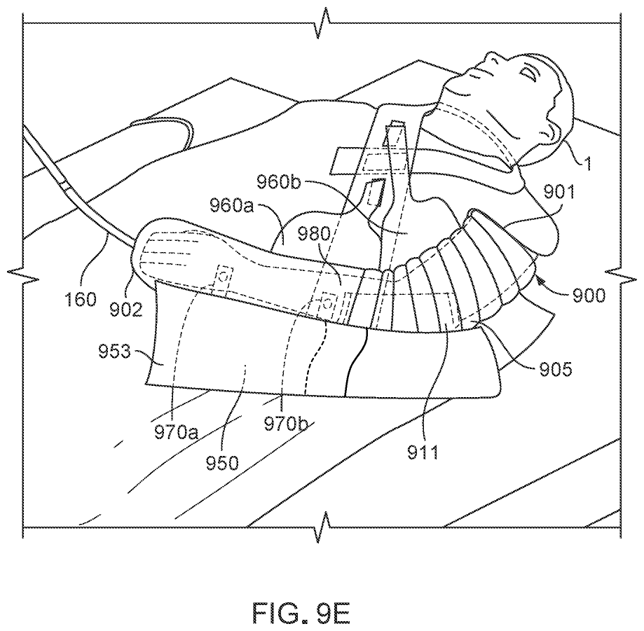

Referring to FIG. 9A, a perspective view of example support sleeve 900 is shown. The support sleeve 900 includes a proximal end 901 that defines an arm opening 901a, and a distal end 902. The wall 903 extends between the proximal end 901 and the distal end 902. The wall includes an anterior side portion 904 and a posterior side portion 905. In some embodiments, the support sleeve 900 is configured to be positioned over a patient's arm such that the anterior side portion 904 of the wall 903 is located generally over the anterior side of the patient's arm, and the posterior side portion 905 of the wall 903 is located generally over the posterior side of the patient's arm. The support sleeve 900 may taper at least partially between proximal end 901 towards an articulated region 908 and/or distal end 902. A larger arm opening 901a may facilitate positioning over a patient's arm, particularly the upper arm/bicep region that is typically larger in circumference than the lower arm or hand region.

The support sleeve 900 includes one or more access ports 906 that facilitate access of the patient's arm through the wall 903. For example, the access port 906 may define an opening through an entire thickness of the wall 903. A medical device may be passed through the access port 906 and into an access location through the patient's skin. In some embodiments, the access port 906 may include a removable liner that at least partially covers an opening defined by the access port 906. The removable liner may be removed at a time of use, such as in the operating room before or after the support sleeve 900 has been positioned over a patient's arm.

In an example embodiment, one or more of the access ports 906 are located to align with a patient's inner wrist when the support sleeve 900 is positioned on the patient's arm. For example, the access port 906 may be located on the anterior side portion 904 of the wall 903 proximate the distal end 902. In this way, a healthcare practitioner may access the radial artery or other vascular feature, for example, via the one or more access ports 906. Alternatively, or additionally, one or more access ports 906 may be located on the anterior side portion 904 of the wall 903 near the proximal end 901, near the inner elbow, etc. (e.g., to facilitate access of other locations on the anterior side of the patient's arm), and/or one or more access ports 906 may be located on the posterior side portion 905 of the wall 903 (e.g., to facilitate access of the posterior side of the patient's arm).

The support sleeve 900 may include one or more adhesive areas on an interior surface of the wall 903. For example, an adhesive 906c on an interior surface of the anterior side portion 904 at least partially surrounds the access port 906. Adhesive 906c promotes sterility by separating the access location of a patient's skin from the remainder of the patient's arm. In some embodiments, adhesive 906c further promotes stability by inhibiting relative movement between the support sleeve 900 and the patient's arm in use.

One or more access ports 906 may be located generally centrally on the anterior side portion 904 of the wall 903 proximate the distal end 902. For example, a central longitudinal axis (a) extending between a proximal end 906a and distal end 906b of the access port 906 may be collinear with a central longitudinal axis of the anterior side portion 904 of the wall 903. A generally centrally located access port 906 may facilitate alignment with the inner wrist surface when the support sleeve is positioned over the patient's arm.

Support sleeve 900 may include one or more features that promote patient comfort and/or protect the patient's arm. In an example embodiment, the support sleeve 900 includes optional padded area 910. Padded area 910 may include a compressible construction that protects and pads the patient's arm, such as padding configured to protect the patient's arm from compression neuropathy and decubitus wound injury, and for comfort. For example, padded area 910 may include locations of increased material thickness and/or may include one or more of foam, closed-cell foam, open-cell foam, compressible non-woven material, rubber, quilted material, laminated material, air pocket(s), water, gel, styrene bead, etc. In some embodiments, padded area 910 may at least partially be filled by an expandable material such that padded material can expand to at least partially conform to the patient's arm.

The padded area 910 may be present over some or all of the support sleeve 900. In an example embodiment, padded area 910 is located on the posterior side portion 905 of the wall 903. Padded area 910 may protect the elbow, triceps brachii, and other posterior features of the patient's arm, particularly when the patient's arm is resting/supported on the posterior side in a supinated position (e.g., during introduction of a medical device at the inner wrist), and/or while an arm is constrained by an arm board or other external structure in contact with the posterior side portion of the support sleeve 900. In an example embodiment, the padded area 910 extends from a proximal location 910a near proximal end 901 to a distal location 910b. Distal location 910b may be selected so that padded area 910 is present at the location of the patient's elbow (e.g., and extends just past the elbow location) when the support sleeve 900 is positioned on the patient's arm in use. Such a configuration may advantageously protect the patient's arm without inhibiting articulation of the support sleeve 900 or patient's arm (e.g., at an elbow location), and/or while facilitating access through access port 906.

The support sleeve 900 may include combinations of two or more materials that protect the patient's arm, and/or multiple padded areas 910 of different material construction. In an example embodiment, a shape-stable, compressible material 111 (FIG. 1) may be located at an elbow region. Compressible material 111 may provide additional padding, and/or support of a patient's elbow. In some embodiments, compressible material 111 may be a foam elbow pad (e.g., open-cell foam, closed-cell foam, etc.), that provides additional protection for the elbow and/or supports the elbow in an articulated position.

Support sleeve 900 is articulable at one or more locations between proximal end 901 and distal end 902. In an example embodiment, the support sleeve 900 includes a preformed articulated region 908. The articulated region 908 simulates an elbow bend and facilitates positioning of the patient's arm in a non-straight orientation. In some embodiments, the articulated region 908 provides a bend such that a lower or distal region of the support sleeve 900 has a first longitudinal axis (A) and the upper or proximal region of the support sleeve has a second longitudinal axis (B) angled relative to the first longitudinal axis (A) (e.g., when the support sleeve 900 is at rest not in use on a patient's arm). The articulated region 908 may thus provide a pre-defined bend that facilitates articulation and may reduce material bunching or gathering when the patient's arm is bent at the elbow while the support sleeve 900 is positioned on the user's arm. Alternatively, or additionally, articulated region 908 may facilitate intuitive positioning on a patient's arm because the support sleeve 900 is at least partially pre-formed in a natural, articulated positon, for example. In various example embodiments, the articulated region 908 includes a preformed bend that defines an angle between 10° and 90°, 30° and 60°, or about 45°. For example, an angle (a) defined between first longitudinal axis (A) and second longitudinal axis (B) may be between 10° and 90°, 30° and 60°, or about 45°. The articulated region 908 is flexible such that a patient's arm may be readily manipulated when the support sleeve 900 is positioned over the patient's arm. For example, the patient's arm can be manipulated during use such that angle (a) is increased or decreased.

The support sleeve 900 may have a generally tubular configuration such that the wall 903 defines a circular or rounded arm opening 901a and/or defines a circular or rounded cross-sectional space between the proximal end 901 and distal end 902. Such a configuration can facilitate positioning of the support sleeve 900 over a patient's arm, while providing an appropriate fit. In various example embodiments, the wall 903 may include a continuous material portion, such as a continuous material portion that is present at both the anterior side portion 904 and the posterior side portion 905. Ends of the continuous material portion may be joined (e.g., by stitching, welding, adhesive, etc.). In some embodiments, the support sleeve 900 may have few or no seams (e.g., such the support sleeve 900 has a seamless construction). Alternatively, or additionally, the wall 903 may include two or more planar sheets of material joined at longitudinal seams, such as longitudinal seam 917. For example, support sleeve 900 may include two or more planar sheets cut to a shape that provides various features described herein. A first sheet may be cut to a shape that includes flexible sheet(s) 940 and an posterior side portion 905, and a second sheet may be cut to a shape that includes attachment devices 960 and an anterior side portion 904. The first and second sheets may be joined (e.g., stitched, welded, etc.) to connect the sheets and define the tubular wall 903 configured to receive a patient's arm. Likewise, the flexible sheet(s) 940 and attachment devices 960 may be portions of the same sheet.

In various example embodiments, wall 903 may have a flexible, liquid-impermeable construction. For example, wall 903 may include one or more layers of polyester, polyethylene, cotton, paper, non-woven, fabric, etc. Alternatively, or additionally, wall 903 may include one of more layers that impart liquid-impermeability, such as a polymer layer. In some embodiments, wall 903 may have a laminated construction of two or more layers. The layers may be microporous to promote breathability while being liquid-impermeable. In some embodiments, the support sleeve 900 may be a stretchable material, such that the wall 903 may stretch and flex to accommodate the patient's arm, while being in close contact and or slight compression against the patient's arm in at least some locations. For example, in use, the wall 903 may be slightly stretched in at least some locations when positioned on the patient's arm. The support sleeve 900 may be a sterile support sleeve that protects the patient from skin flora and the external environment.

The support sleeve 900 may have one or more securing features that promote a secure fit on the patient's arm, and/or inhibit relative movement between the support sleeve 900 and the patient's arm during use (e.g., relative rotation and/or longitudinal translation). For example, the support sleeve may include one or more securing features 909 near proximal end 901. Securing features 909 may include one or more of an elasticized region, strap, tie, hook-and-loop fastener, adjustment mechanism, etc. that promotes a secure fit around the patient's upper arm. In an example embodiment, the securing feature 909 does not extend around an entire perimeter of the wall 903. One or more securing features 909 may be located elsewhere between proximal end 901 and distal end 902, such as above the articulated region 908, below the articulated region 908, proximate the access port 906, etc.

In various example embodiments, the support sleeve 900 may include one or more finger and/or thumb portions configured to receive an individual finger/thumb. Such a configuration may facilitate positioning the support sleeve, and/or promote patient comfort. In an example embodiment, the support sleeve includes a pulse oximeter 907 in the sleeve configured to monitor (e.g., continuously) the patient's hand oxygen supply.

The support sleeve 900 may be configured to at least partially stabilize a patient's arm in a selected position. In an example embodiment, the support sleeve 900 optionally includes one or more attachment devices 970a, 970b, and 960a, 960b that facilitate engagement with a substrate, such as a complementary stabilization substrate in the operating environment. For example, attachment devices 960a, 960b includes areas 963a, 963b (and attachment devices 970a and 970b have areas 973a and 973b, respectively) that, when brought into contact with another surface, engages the surface to limit relative movement between the support sleeve 900 and the surface. In various example embodiments, the attachment devices 960a, 960b, 970a and 970b include one or more of adhesives, hook-and-loop fasteners, self-adherent non-woven materials, self-adherent polymer materials, highly frictional polymer materials, coadhesive materials, etc. In some embodiments, the areas 963, 973a and 973b of the attachment devices may be exposed (e.g., by removing a liner covering an adhesive), and the attachment device brought into contact with a stabilization substrate (not shown in FIG. 9A), such as a patient's skin, sterile surgical drape, belt, neckband, strap, other stabilization substrate, etc. In various embodiments, only areas 963a, 963b, 973a, 973b of attachment devices 960, 970 include one or more of adhesives, hook-and-loop fasteners, self-adherent non-woven materials, self-adherent polymer materials, highly frictional polymer materials, coadhesive materials, etc. Alternatively, or additionally, the entire attachment devices 960, 970 may include such materials.

As shown in FIG. 9A, attachment device 960 may comprise a flexible sheet material that is attached to the support sleeve 900 along or near end 964a. In one embodiment, the attachment device may comprise a wide first end 964a at the attachment line and then narrow towards a second end 964b at least partially along its length 962. The attachment device 960 optionally may be folded along its length 962 prior to use, e.g., at lines 965a, 965b, 965c, 965d, etc. or the attachment device 960 may be rolled. For example, the attachment device 960 may be folded and/or rolled at a time of manufacture, and unfolded/unrolled before positioning on a patient's arm or during use after positioning on a patient's arm.

As shown in FIG. 9A, attachment device 960 may comprise a flexible sheet material 940 (or multiple narrower sheet materials 940a, 940b that may overlap, e.g., at the articulated region of support sleeve 900) that is attached to the support sleeve 900 along or near edge 941. In one embodiment, flexible sheet material 940 is initially folded along attachment line 941 such that a first major surface 953 of flexible sheet material 940 is adjacent the posterior major surface of support sleeve 900. In use, support sleeve 900 may be provided in a sterilized condition. The patient's arm may be inserted into the device while flexible sheet 940 is folded along edge 941. Then, for example, the covered arm of the patient, with the palm faced upwards, may be placed atop the patient with the flexible sheet material 940 lying adjacent the patient's body. Attachment devices 970a and 970b may be secured to the patient (or to a stabilization substrate) and the arm rotated over to assume a palm down second position. Flexible sheet 940 can then be unfolded to expose a still sterile inner major surface 953 of the sheet.

Flexible sheet 940, when in its initial folded configuration, protects the sterility of surfaces 905 and 953, and these major surfaces, which become exposed when the patient's arm is placed in the “across the belly” second position shown in FIG. 9C, create a sterile field. In an example embodiment, first and second sheet materials 940a, 940b, are portions of different sheet materials from one another that are attached to one or more portions of support sleeve 900. For example, first sheet material 940a may be cut from a first sheet of material that also defines posterior side portion 905, and second sheet material 940b may be cut from a second sheet of material that also defines anterior side portion 904. Such a construction can facilitate manufacturing of support sleeve 900, and promote articulation of the first and second sheet materials 940a, 940b relative to one another (e.g., during movement of the patient's arm).

Referring to FIGS. 9B and 9C, the example support sleeve 900 is shown in use in a first position (FIG. 9B) and a second position (FIG. 9C), which may be similar to the positions described above with reference to FIGS. 3-6B, for example. In the first position, the arm is in a supinated position in which the anterior side of the patient's arm is facing upwards. The inner-wrist is thus facing upwards and accessible (e.g., via access port 906) by a healthcare practitioner. A medical device may thus be introduced into the patient's arm proximate the inner wrist, such as to access the radial artery or other feature of the patient. Attachment devices 970a and/or 970b may be secured to stabilization substrate 150 or a lower portion of shoulder strap 955b. When the arm is rotated into the second position as shown in FIG. 9C, attachment device 960 may be attached to the stabilization substrate 150 or an upper portion of shoulder straps 955a and/or 955b.

Referring to FIGS. 9D and 9E, example support sleeve 900 is shown in use with a stabilization substrate in the form of a neck bib 957. Stabilization substrate 957 may include a length of material configured to extend around a patient's neck and/or attach to itself. An attachment feature 958a at a first end of the stabilization substrate 957 may attach to a complementary feature 958b along the length of the stabilization substrate 957 to maintain the stabilization substrate 957 in a desired position around the patient's neck. The neck bib includes one or more features attachable with support sleeve 900 to stabilize the support sleeve in a selected position.

In an example embodiment, the stabilization substrate 957 may be packaged in a sterile condition. At least a portion of the stabilization substrate 957 (e.g., a portion that is positioned under, or attached to, the support sleeve during use) may be packaged in a folded or rolled configuration. In some embodiments, the stabilization substrate 957 may be secured on a patient by extending around the patient's neck while at least partially in the folded or rolled condition. Such a configuration may promote sterility and provide an additional sterile field (e.g., that is maintained sterile until unfolded/unrolled).

The example support sleeve 900 is shown in a first position (FIG. 9D) and a second position (FIG. 9E), which may be similar to the positions described above with reference to FIGS. 3-9C, for example. In the first position, the arm is in a supinated position in which the anterior side of the patient's arm is facing upwards. The inner-wrist is thus facing upwards and accessible (e.g., via access port 906) by a healthcare practitioner. A medical device may be introduced into the patient's arm proximate the inner wrist, such as to access the radial artery or other feature of the patient.

When the arm is in the first, supinated position (e.g., before attachment with stabilization substrate 957), an attachment region 959 of the stabilization substrate 957 may be at least partially folded or rolled upon itself. In such a configuration, the sterility of the folded or rolled surfaces of the stabilization substrate 957 may be maintained. The attachment region 959 may be unrolled/unfolded to facilitate attachment with medical support sleeve 900. For example, the attachment region 959 may be unrolled/unfolded such, when the patient's arm is rotated from the first, supinated position to the second pronated position, attachment device(s) 960 may be attached to the stabilization substrate 957, such as at attachment features 959a of attachment region 959.

In various example embodiments, the support sleeve 900 (e.g., attachment device(s) 960) includes one or more attachment devices, such as one or more of an adhesive, hook-and-loop fastener, snaps, straps, ties, frictional materials, self-adherent non-woven materials, self-adherent polymer materials, coadhesive materials, highly frictional polymer, etc. The stabilization substrate 957 may likewise include one or more complementary attachment devices, such as complementary adhesive, hook-and-loop fastener, snaps, straps, ties, frictional materials, self-adherent non-woven materials, self-adherent polymer materials, coadhesive materials, highly frictional polymer, etc.

Engagement between the support sleeve 900 and the stabilization substrate 957 inhibits relative movement between the support sleeve 900 and the stabilization substrate 957, and promotes support of the patient's arm in the selected position (e.g., shown in FIG. 9E). The support sleeve 900 facilitates positioning of the patient's arm in one or more selected positions based on the healthcare practitioner's preferences, patient anatomy, the medical procedure being performed, etc. The second position is not limited to an orientation in which the elbow is bent at about 90°. The healthcare may select an appropriate angle, including angles that allow the wrist to rest at a location near the inguinal region. The material and construction of the support sleeve 900 allow articulation of the patient's arm between multiple positions, while supporting the patient's arm in the selected position at least partially by engagement with stabilization substrate 957.

While this specification contains many specific implementation details, these should not be construed as limitations on the scope of any technology or of what may be claimed, but rather as descriptions of features that may be specific to particular embodiments. Certain features that are described in this specification in the context of separate embodiments can also be implemented in combination in a single embodiment in part or in whole. Conversely, various features that are described in the context of a single embodiment can also be implemented in multiple embodiments separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Particular embodiments of the subject matter have been described. Other embodiments are within the scope of the following claims.

1.-35. (canceled)

36. A medical support sleeve system, comprising:
a proximal end that defines an arm opening;
a distal end;
a wall that extends between the proximal end and the distal end;
an access port located proximate the distal end; and
an expandable sterile drape portion configured to be expanded to cover a portion of a patient's body, the expandable sterile drape portion adjustable from an unexpanded condition in which the expandable sterile drape is retained proximate the wall to an expanded condition in which the expandable sterile drape extends away from the wall while the medical support sleeve is positioned over a patient's arm.

37. The medical support sleeve system of claim 36, wherein the expandable sterile drape portion is configured to extend from an ulnar region of a patient's arm when in the expanded condition.

38. The medical support sleeve system of claim 36, wherein the wall includes an anterior side portion and a posterior side portion.

39. The medical support sleeve system of claim 38, wherein the access port is located on the anterior side portion of the wall.

40. The medical support sleeve system of claim 36, comprising a pulse oximeter attached to the medical support sleeve proximate the distal end.

41. The medical support sleeve system of claim 36, comprising an attachment device configured to attach the wall of the support sleeve to a substrate.

42. The medical support sleeve system of claim 41, wherein the attachment device comprises an adhesive.

43. The medical support sleeve system of claim 41, wherein the attachment device is selected from the group consisting of hook-and-loop fastener, snaps, hooks, straps, ties, self-adherent non-woven material, etc.

44. The medical support sleeve system of claim 41, wherein the sleeve comprises a central longitudinal axis that extends through the access port, and the attachment device comprises a first strap laterally offset on a first side of the central longitudinal axis, and a second strap laterally offset on a second side of the central longitudinal axis opposite from the first side of the central longitudinal axis.

45. The medical support sleeve system of claim 44, wherein the support sleeve is manipulatable from a pronated position towards a supinated position while the support sleeve is attached to the substrate by at least one of the first strap or the second strap.

46. The medical support sleeve system of claim 44, comprising an adhesive at least partially surrounding the access port on an interior surface, the adhesive configured to adhere to a patient's skin when the wall is positioned around a patient's arm.

47. The medical support sleeve system of claim 36, wherein the wall is made from a fluid impermeable material.

48. The medical support sleeve system of claim 36, wherein the support sleeve includes a preformed articulated region that defines an angle between 10° and 90°.

49. The medical support sleeve system of claim 48, wherein when the support sleeve is positioned on a patient's arm during use, the preformed articulated region is configured to be located at an elbow location of a patient's arm while the access port is located at an inner wrist location of the patient's arm.

50. The medical support sleeve system of claim 41, further comprising a stabilization substrate, wherein the attachment device is located on the stabilization substrate.

51. A medical support sleeve system, comprising:
a proximal end that defines an arm opening;
a distal end;
a wall that extends between the proximal end and the distal end;
an access port located proximate the distal end; and
an expandable sterile drape portion configured to be expanded to cover a portion of a patient's body, the expandable sterile drape portion adjustable from a first condition in which a major surface of the expandible sterile drape is maintained in a sterile condition, and a second condition in which the the expandable sterile drape extends away from the wall.

52. The medical support sleeve system of claim 51, wherein the wall includes a posterior wall portion and an anterior wall portion, and wherein the major surface is maintained proximate the posterior wall portion in the first condition and extends outwardly away from the posterior wall portion in the second condition.

53. The medical support sleeve system of claim 52, wherein the access port is located on the anterior side portion of the wall.

54. The medical support sleeve system of claim 36, comprising an attachment device configured to attach the wall of the support sleeve to a substrate.

55. The medical support sleeve system of claim 44, wherein the attachment device includes a first strap and a second strap, and the support sleeve is manipulatable from a pronated position towards a supinated position while the support sleeve is attached to the substrate by at least one of the first strap or the second strap.
